(19) United States
(12) Patent Application Publication
(10) Pub. No.: US 2016/0183782 A1
(43) Pub. Date: Jun. 30, 2016

(54) OCT SURGICAL VISUALIZATION SYSTEM WITH MACULAR CONTACT LENS

(71) Applicant: NOVARTIS AG, Basel (CH)

(72) Inventors: Lingfeng Yu, Lake Forest, CA (US); Vadim Shofman, Fort Worth, TX (US)

(21) Appl. No.: 14/584,830

(22) Filed: Dec. 29, 2014

(51) Int. Cl. A61B 3/10; A61B 3/13; A61N 5/06; A61B 3/14; A61F 9/008; A61B 19/00; A61B 3/00

(52) U.S. Cl. CPC A61B 3/102; A61B 19/5223; A61B 3/13; A61B 3/0008; A61B 3/14; A61B 3/1025; A61F 9/00823; A61N 5/06; A61F 2009/00863

(57) ABSTRACT

An ophthalmic visualization system can include an ocular lens positioned between a macular contact lens coupled to a procedure eye and a surgical microscope. The ocular lens can guide a light beam through the macular contact lens and into the procedure eye, and in combination with the macular contact lens generate an intermediate image of the procedure eye at an image plane between the procedure eye and the surgical microscope. The system can include a reduction lens positioned in the optical path between the surgical microscope and the ocular lens. The reduction lens and/or ocular lens can align a focus plane of the surgical microscope with the image plane. A method of visualizing a procedure eye in an ophthalmic procedure can include positioning an ocular lens and a reduction lens between a macular contact lens and a surgical microscope; and scanning the procedure eye with a light beam.

### BACKGROUND

1. Technical Field

Embodiments disclosed herein are related to ophthalmic visualization systems. More specifically, embodiments described herein relate to ophthalmic procedures utilizing a macular contact lens coupled to a procedure eye. The ophthalmic visualization system can simultaneously scan a target region within the procedure eye with a light beam, such as an optical coherence tomography (OCT) scanning beam, and directly view the target region with a surgical microscope.

2. Related Art

Some types of ophthalmic surgery involve the use of a macular contact lens. These procedures can include macular surgeries to treat membrane peeling, a macular hole, and/or an epiretinal membrane, among other ophthalmic disorders. During the procedure, a surgeon views the part of the patient's eye being operated on through a surgical microscope. With the macular contact lens coupled to the eye, the surgeon sees an upright virtual image of the target region behind the macular contact lens. The macular contact lens provides the surgeon with better lateral resolution and depth perception compared to wide-field viewing systems such as a binocular indirect ophthalmomicroscope (BIOM) type or wide-field indirect contact lens. However, the macular contact lens provides a relatively narrow field of view compared to the wide-field viewing systems.

Optical coherence tomography (OCT) can be a noninvasive, high resolution cross-sectional imaging modality. Conventional microscope-integrated OCT systems can be designed for wide-field viewing systems and, therefore, are difficult to implement with the narrow field of view of a macular contact lens. For example, with the macular contact lens in place, OCT imaging can be compromised because the range of the OCT scanning beam is limited by the pupil of the patient eye. If the OCT scanning beam pivots relatively far from the pupil plane, even slight changes in the direction cause the OCT scanning beam to terminate at opaque portions of the eye.

Accordingly, there remains a need for improved devices, systems, and methods that facilitate implementation of OCT imaging with a macular contact lens while preserving the ability of a surgeon to directly view a target region through the surgical microscope, by addressing one or more of the needs discussed above.

### SUMMARY

The presented solution fills an unmet medical need with a unique solution to provide simultaneous direct viewing and OCT imaging with a macular contact lens positioned on the procedure eye. An ocular lens and a reduction lens can be positioned between a surgical microscope and the procedure eye. The ocular lens can allow an OCT scanning beam to pivot at the pupil and reach a wider field of view within the procedure eye. The reduction lens can allow a surgeon to directly view a target region in the procedure eye clearly without refocusing the optics of the surgical microscope.

Consistent with some embodiments, an ophthalmic visualization system can be provided. The system includes: an ocular lens configured to be positioned in an optical path between a macular contact lens coupled to a procedure eye and a surgical microscope, wherein the ocular lens is configured to guide a light beam through the macular contact lens and focus into the procedure eye; and generate an intermediate image plane associated with light reflected from the procedure eye, the image plane being positioned between the procedure eye and the surgical microscope; and a reduction lens positioned in the optical path between the surgical microscope and the ocular lens, wherein the reduction lens is configured to align a focus plane of the surgical microscope with the intermediate image plane.

Consistent with some embodiments, a method of visualizing a procedure eye in an ophthalmic procedure can be provided. The method includes: positioning an ocular lens in an optical path between a macular contact lens coupled to the procedure eye and a surgical microscope such that an intermediate image plane associated with light reflected from the procedure eye is generated between the procedure eye and the surgical microscope; positioning a reduction lens in the optical path between the surgical microscope and the ocular lens such that a focus plane of the surgical microscope is aligned with the intermediate image plane; and scanning the procedure eye with a light beam including guiding the light beam through the macular contact lens and into the procedure eye using the ocular lens.

Additional aspects, features, and advantages of the present disclosure will become apparent from the following detailed description.

### BRIEF DESCRIPTION OF THE DRAWINGS

FIG. 1 is a diagram illustrating an ophthalmic visualization system.

FIG. 2 is a diagram illustrating an ophthalmic visualization system.

FIG. 3 is a diagram illustrating a portion of an ophthalmic visualization system.

FIG. 4a is a diagram illustrating an ocular lens.

FIG. 4b is a diagram illustrating an ocular lens.

FIG. 5 is a diagram illustrating a portion of an ophthalmic visualization system.

FIG. 6 is a diagram illustrating a portion of an ophthalmic visualization system.

FIG. 7 is a diagram illustrating an ophthalmic visualization system.

FIG. 8 is a diagram illustrating an ophthalmic visualization system.

FIG. 9 is a flow diagram illustrating a method of visualizing a procedure eye in an ophthalmic procedure.

In the drawings, elements having the same designation have the same or similar functions.

### DETAILED DESCRIPTION

In the following description specific details are set forth describing certain embodiments. It will be apparent, however, to one skilled in the art that the disclosed embodiments may be practiced without some or all of these specific details. The specific embodiments presented are meant to be illustrative, but not limiting. One skilled in the art will realize that other material, although not specifically described herein, is within the scope and spirit of this disclosure.

The present disclosure describes devices, systems, and methods that facilitate and optimize simultaneous wide-field OCT imaging and direct visualization using a surgical microscope with a macular contact lens in place. An ocular lens and a reduction lens can be provided between the surgical microscope and the procedure eye. The ocular lens and the reduction lens can work together with the macular contact lens to facilitate both OCT imaging and direction visualization without changing the configuration of the visualization system. The ocular lens can locate a pivot point of the OCT scanning beam at the pupil of the procedure eye to enable a wider field of view for OCT imaging. The ocular lens, in combination with the macular contact lens, can generate an intermediate image plane positioned between the surgical microscope and the procedure eye.. The reduction lens can shift the location of a focus plane of the surgical microscope into alignment with the intermediate image plane such that an operator, such as a surgeon or other medical professional, can see the target region clearly without adjusting the distance between the procedure eye and the surgical microscope, or refocusing the microscope optics. The ocular lens and the reduction lens can be selectively moved such that an operator can switch between a microscope viewing only mode and a simultaneous scanning and microscope viewing mode. The target region can be clearly viewed through the surgical microscope in both modes without making focus adjustments.

The devices, systems, and methods of the present disclosure provide numerous advantages, including: (1) providing microscope-integrated OCT imaging with a macular contact lens; (2) permitting simultaneous direct/microscope viewing and OCT imaging with a macular contact lens; (3) permitting direct viewing and OCT imaging without changing the configuration of elements in the visualization system; (4) allowing easy switching between a direct viewing only mode and simultaneous direct viewing and scanning mode; (5) simplified surgical workflow that does not require adjusting the distance between procedure eye and the surgical microscope, or refocusing the surgical microscope; (6) wider field of view of direct visualization and OCT imaging with comparable or better lateral resolution; (7) decreased total lens aberration with the addition of the ocular lens and the reduction lens to compensate for any aberrations from the macular contact lens only.

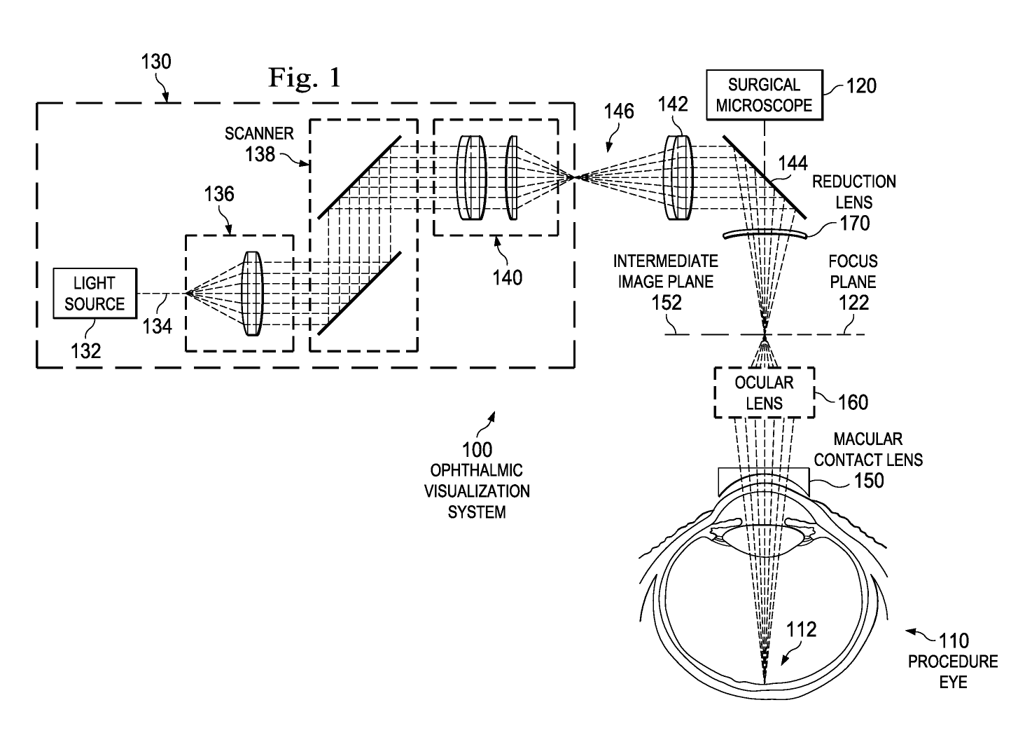

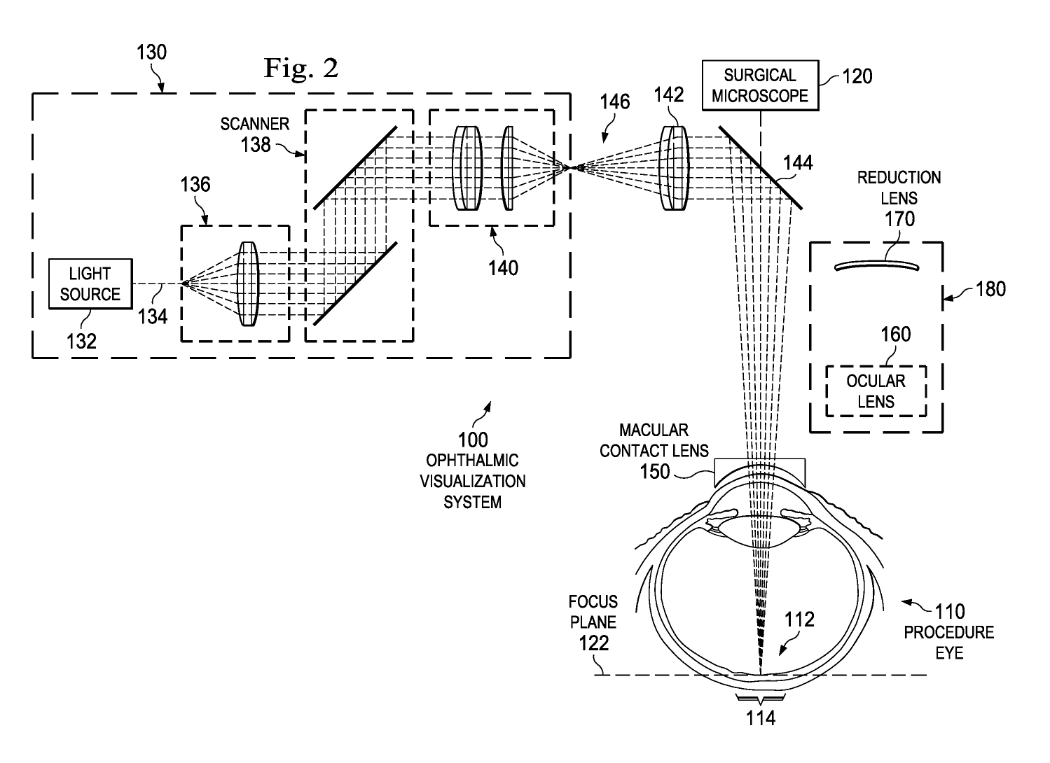

Referring to FIGS. 1 and 2, shown therein is an ophthalmic visualization system 100. The ophthalmic visualization system 100 can include an ocular lens 160. The ocular lens 160 can be configured to be positioned in an optical path between a macular contact lens 150 coupled to a procedure eye 110 and a surgical microscope 120. The ocular lens 160 can also be configured to guide a light beam 146 through the macular contact lens 150 and into the procedure eye 110. The ocular lens 160 can be further configured to generate an intermediate image plane 152 associated with light reflected from the procedure eye 110. The intermediate image plane 152 can be positioned between the procedure eye 110 and the surgical microscope 120. The ophthalmic visualization system 100 can also include a reduction lens 170 positioned in the optical path between the surgical microscope 120 and the ocular lens 160. The reduction lens 170 can be configured to align a focus plane 122 of the surgical microscope 120 with the intermediate image plane 152. As described in more detail below, the ocular lens 160 and the reduction lens 170 are selectively positionable within the optical path between the surgical microscope 120 and the procedure eye 110. In FIG. 1, the ocular lens 160 and the reduction lens 170 are positioned in the optical path. In FIG. 2, the ocular lens 160 and the reduction lens 170 are removed from the optical path.

The ophthalmic visualization system 100 can be used during an ophthalmic procedure with the macular contact lens 150 coupled to the procedure eye 110. The macular contact lens 150 can include one or more optical components, such as a biconcave lens, biconvex lens, convex-concave lens, plano concave lens, plano convex lens, positive/negative meniscus lens, aspheric lens, converging lens, diverging lens, and other suitable lenses. For example, the macular contact lens 150 can be the GRIESHABER® DSP Aspheric Macular Lens available from Alcon, Inc. The macular contact lens 150 can be embedded in a stabilizing mechanism. The stabilizing mechanism can be configured to stabilize the macular lens 150 relative to the procedure eye 110. To that end, the stabilizing mechanism can include one or more of a trocar, a counter weight, a friction-based system, and an elastic system.

The ophthalmic visualization system 100 can scan the target region 112 using a beam delivery system 130 while simultaneously allowing direct viewing of the target region 112 using the surgical microscope 120. The target region 112 can include the retina, macula, foveola, fovea centraalis, para fovea, perifovea, optic disc, optic cup, one of more layers of the retina, vitreous, vitreous body, etc.

The ophthalmic visualization system 100 can include an optical path associated with the light beam 146 of the beam delivery system 130. The light beam 146 scans the target region 112 within the procedure eye 110. The optical path of the light beam 146 can extend between the beam delivery system 130 and the procedure eye 110. The beam delivery system 130 can include at least one light source 132 configured to generate the light beam 146. For example, the beam delivery system 130 can include one light source to generate a diagnostic light beam, one light source to generate a treatment light beam, and one light source to generate an illumination light beam. For example, the light source 132 can be configured to generate the diagnostic light beam, the treatment light beam, and the illumination light beam. In that regard, the light source 132 can be part of a treatment beam delivery system, such as a laser beam delivery system, a photocoagulation system, a photodynamic therapy system, a retinal laser treatment system.

The light source 132 can be part of an illumination beam delivery system. The illumination beam delivery system can be configured to provide light to illuminate the interior of the procedure eye 110, including the target region 112, during the surgical procedure. The illumination beam delivery system can be configured to output a red illumination light, a blue illumination light, a green illumination light, a visible illumination light, a near infrared illumination light, an infrared illumination light, other suitable light, and/or combinations thereof.

The light source 132 can be part of a diagnostic imaging system, such as an OCT imaging system, a multispectral imaging system, a fluorescence imaging system, a photo-acoustic imaging system, a confocal scanning imaging system, a line-scanning imaging system, etc. For example, the light beam can be part of an OCT scanning beam.

The light source 132 can have an operating wavelength in the 0.2-1.8 micron range, the 0.7-1.4 micron range, and/or the 0.9-1.1 micron range. The OCT system can be configured to split an imaging light received from a light source into an imaging beam that is directed onto target biological tissue and a reference beam that can be directed onto a reference mirror. The OCT system can be a Fourier domain (e.g., spectral domain, swept-source, etc.) or a time domain system. The OCT system can be further configured to receive the imaging light reflected from the target biological tissue. The interference pattern between the reflected imaging light and the reference beam can be utilized to generate images of the target biological tissue. Accordingly, the OCT system can include a detector configured to detect the interference pattern. The detector can include a balanced photo-detector, a InGaAs PIN detector, a InGaAs detector array, a Si PIN detector, Charge-Coupled Detectors (CCDs), pixels, or an array of any other type of sensor(s) that generate an electric signal based on detected light. Further, the detector can include a two-dimensional sensor array and a detector camera. A computing device can process the data acquired by the OCT system to generate a two-dimensional or three-dimensional OCT image.

The beam delivery system 130 can include a beam guidance system 134, including an optical fiber and/or free space, configured to guide the light beam 146 from the light source 132. The beam delivery system 130 can include a collimator 136 that is configured to receive the light beam 146 from the beam guidance system 134 and collimate the light beam 146.

The beam delivery system 130 can include a scanner 138 configured to receive the light beam 146 from the collimator 136 and/or the beam guidance system 134, and scan the light beam 146. For example, the scanner 138 can be configured to receive the diagnostic light beam from the beam guidance system and scan the diagnostic light beam across a pattern. The scanner 138 can be configured instead or additionally to receive the treatment light beam from the beam guidance system and scan the treatment light beam across a pattern. The scanner 138 can be configured to scan the light beam 146 over any desired one-dimensional or two-dimensional scan patterns, including a line, a spiral, a raster, a circular, a cross, a constant-radius asterisk, a multiple-radius asterisk, a multiply folded path, and/or other scan patterns. The scanner 138 can include one or more of a scanning minor, a micro-mirror device, a MEMS based device, a deformable platform, a galvanometer-based scanner, a polygon scanner, and/or a resonant PZT scanner. The scanner 138 can direct the light beam 146 through one or more focusing and/or zoom lenses 140 and an objective lens 142. The focusing and/or zoom lenses 140 and the objective lens 142 can be fixed or adjustable, and can define a depth of focus of the light beam 146 within the procedure eye 110.

The beam delivery system 130 can also include a beam coupler 144 configured to redirect the light beam 146 towards the ocular lens 160. The beam coupler 144 can include a dichroic mirror, a notch filter, a hot minor, a beamsplitter and/or a cold mirror. The beam coupler 144 can be configured to combine the light utilized by the surgical microscope 120 to visualize the procedure eye 110 with the light beam 146. The field of view of the light beam 146 and the surgical microscope 120 can overlap completely, overlap partially, or not overlap at all. The beam coupler 144 can be configured to reflect light in the wavelength range of the light beam 146 while allowing the light of a different wavelength range (such as a visible range from about 470 nm to about 660 nm) reflected from the procedure eye 110 to pass therethrough to the surgical microscope 120.

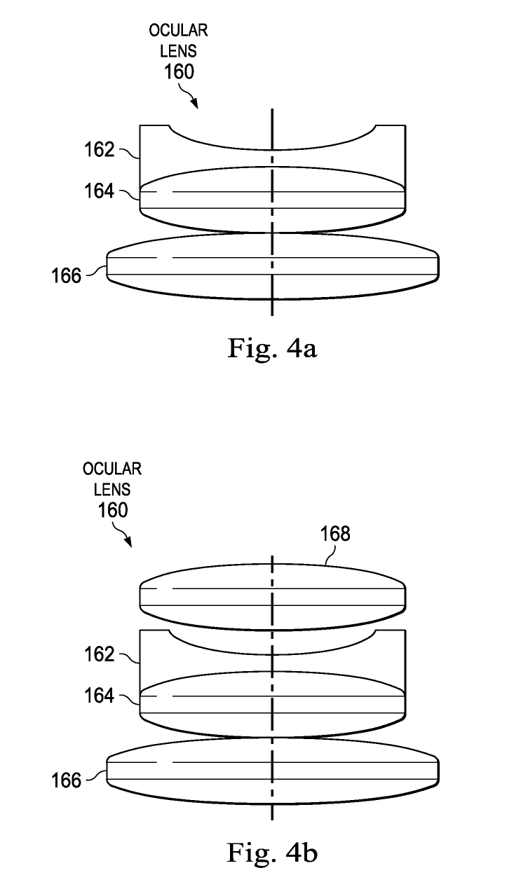

As noted above, the ophthalmic visualization system 100 can include the ocular lens 160. Embodiments of the ocular lens 160 are described in greater detail with respect to FIGS. 4a and 4b. The ocular lens 160 can be implemented as a direct or indirect, contact or non-contact lens. For example, the ocular lens 160 can be a non-contact lens that is spaced from the procedure eye 110 and/or the macular contact lens 150. The ocular lens 160 can include one or more optical components, such as a biconcave lens, biconvex lens, convex-concave lens, plano concave lens, plano convex lens, positive/negative meniscus lens, aspheric lens, converging lens, diverging lens, other suitable lenses, and/or combinations thereof. The ocular lens 160 can be configured to guide the light beam 146 through the macular contact lens 150 and into the target region 112 of the procedure eye 110. The light beam 146 scans the target region 112 for diagnostic imaging, treatment, and/or illumination.

The ophthalmic visualization system 100 can also include an optical path associated with the light reflected from procedure eye 110 and received at the surgical microscope 120. The optical path of the light extends between the surgical microscope 120 and the procedure eye 110. The light forms an optical image of the target region 112. An operator can view the optical image of the target region 112 using the surgical microscope 120. The surgical microscope 120 can include one or more lenses, such as focusing lens(es), zoom lens(es), and an objective lens, as well as minors, filters, gratings, and/or other optical components that comprise an optical train. The surgical microscope 120 can be any microscope suitable for use in an ophthalmic procedure.

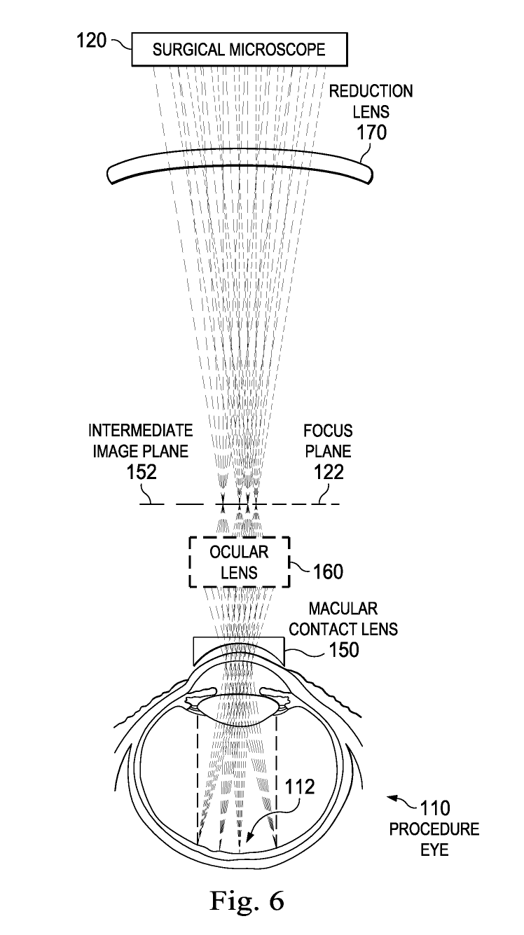

In the ophthalmic visualization system 100 of FIG. 1, the light reflected/scattered from the procedure eye 110 forms a real image of the target region 112 along the intermediate image plane 152. The intermediate image plane 152 can be positioned between the surgical microscope 120 and the procedure eye 110, between the surgical microscope 120 and the macular contact lens 150, between the surgical microscope 120 and the ocular lens 160, etc. The location of the intermediate image plane 152 can change based on the relative positioning and type of the optical components in the optical path between the procedure eye 110 and the reduction lens 170, such as the macular contact lens 150 and the ocular lens 160. As described in greater detail with respect to FIG. 6, the ocular lens 160 can be configured to shift the location of the intermediate image plane 152.

When the intermediate image plane 152 and the focus plane 122 of the surgical microscope 120 are aligned, the operator can view the target region 112 clearly through surgical microscope 120. During an ophthalmic procedure, the procedure eye 110 generally remains at a fixed distance from the surgical microscope 120. This distance can be described as the working distance of the surgical microscope 120. Even though the working distance can influence whether the optics of the surgical microscope 120 are focused, moving the procedure eye 110 closer to or farther from the surgical microscope 120 during the ophthalmic procedure can usually be inconvenient and disfavored. An operator can focus the surgical microscope 120 along the focus plane 122 by using various coarse focus and fine focus controls to change the relative positioning of the optical components of the surgical microscope 120. Because working distance generally does not change, the operator need only focus the surgical microscope 120 once, typically at the beginning of the surgical procedure. Adjusting the focus controls of the surgical microscope 120 during the ophthalmic procedure can be cumbersome. So long as the location of the intermediate image plane 152 does not shift, an operator can see the target region 112 clearly using the surgical microscope 120 for the duration of the ophthalmic procedure.

The relative positioning and type of the optical components in the optical path between the surgical microscope 120 and the reduction lens 170, such as the beam coupler 144, can influence the location of the focus plane 122. As described in greater detail with respect to FIG. 6, the reduction lens 170 can shift the location of the focus plane 122 to bring it into alignment with the intermediate image plane 152. The reduction lens 170 can include one or more optical components, such as a biconcave lens, biconvex lens, convex-concave lens, plano concave lens, plano convex lens, positive/negative meniscus lens, aspheric lens, converging lens, diverging lens, liquid crystal lens, diffractive lens, other suitable lenses, and/or combinations thereof.

As illustrated in FIG. 1, the ocular lens 160 and the reduction lens 170 can be separate components. As illustrated in FIG. 2, the ocular lens 160 and the reduction lens 170 can be integrated into an optical block 180. The ocular lens 160, the reduction lens 170, and/or the optical block 180 can have a defined optical/optomechanical relationship to the surgical microscope 120. For example, when the ocular lens 160 and the reduction lens 170 are separate components, each one can be configured to be movably coupled to the surgical microscope 120 and/or the other of the ocular lens 160 and the reduction lens 170. For example, when the ocular lens 160 and the reduction lens 170 are integrated into the optical block 180, the optical block 180 can be configured to be movably coupled to the surgical microscope 120. The ocular lens 160, the reduction lens 170, and/or the optical block 180 are configured to selectively translate, rotate, pivot, or otherwise move into and out of the optical path between the surgical microscope 120 and the procedure eye 110. Direct or indirect coupling among the surgical microscope 120, the ocular lens 160, the reduction lens 170, and/or the optical block 180 can include one or more of a suspension system, a mechanical frame, a protruding arm, a conical structure, a magnetic member, an elastic member, and a plastic member. The operator can move the ocular lens 160, the reduction lens 170, and/or the optical block 180 manually, or using a motorized actuator or other mechanical and/or electromechanical controller. When the ocular lens 160, the reduction lens 170, and/or the optical block 180 is not position in the optical path, the focus plane 122 of the surgical microscope 120 can be positioned along the target region 112. Thus, the operator can view the target region 112 clearly using the surgical microscope with the macular contact lens 150 in place.

Because the ocular lens 160, the reduction lens 170, and/or the optical block 180 are selectively movable into and out of the optical path, the ophthalmic visualization system 100 can be selectively implemented either for direct/microscope viewing only or for simultaneous scanning and direct/microscope viewing. With the ocular lens 160 and the reduction lens 170 removed from the optical path, the operator can view the target region 112 using the surgical microscope 120 with the macular contact lens 150 in place. With the ocular lens 160 and the reduction lens 170 positioned in the optical path, the target region 112 can be simultaneously scanned by the beam delivery system 130 and directly viewed with the surgical microscope 120.

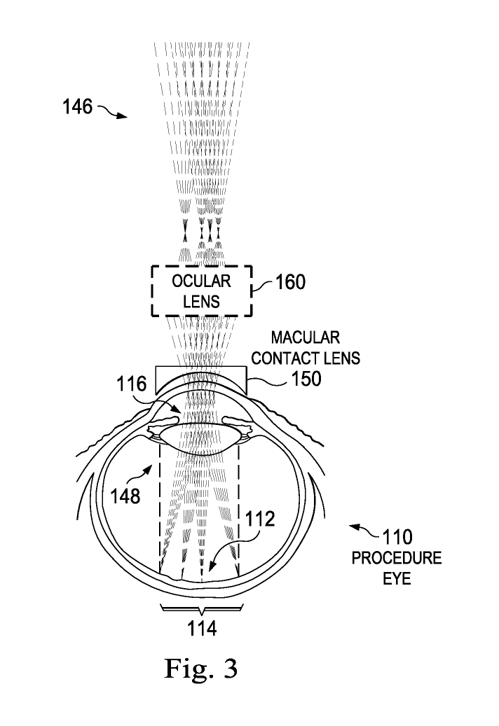

FIG. 3 illustrates a portion of the ophthalmic visualization system 100 associated with the light beam 146 that scans the target region 112 of the procedure eye 110. From the beam coupler 144 (FIGS. 1 and 2), the light beam 146 can be guided by the ocular lens 160 through the macular contact lens 150 and into the procedure eye 110. The light beam 146 can converge beyond the ocular lens 160 and can be focused at the target region 112.

With the ocular lens 160 positioned in the optical path as illustrated in FIG. 3, a pivot point 148 of the light beam 146 can be located at the pupil 116. The pivot point 148 can describe the location in the optical path where the direction of the light beam 146 changes as the target region 112 is scanned. A wider field of view 114 in the target region 112 can be scanned with the pivot point 148 located at or near the pupil 116. When the pivot point 148 is located closer to the pupil 116, the direction of the light beam 146 can be changed by a relatively greater amount without the light beam 146 encountering opaque portions of the eye, such as the iris. In contrast, when the ocular lens 160 is removed from the optical path as illustrated in FIG. 2, the pivot point of the light beam 146 can be located at the beam coupler 144, which is relatively far from the target region 112. As a result, even slight changes in the direction of the light beam 146 can cause the light beam 146 to encounter opaque portions of the eye, resulting in a relatively narrow field of view 114. Thus, the ocular lens 160 allows for more of the target region 112 to be scanned by the light beam 146 because of the wider field of view 114 provided by the ocular lens 160 (FIG. 3).

FIGS. 4a and 4b illustrate embodiments of the ocular lens 160. Embodiments of the ocular lens 160 can include one, two, three, four, or more individual components. In that regard, the ocular lens 160 can be an ocular lens assembly. FIG. 4a shows the ocular lens 160 with three lenses 162, 164, and 166. FIG. 4b shows the ocular lens 160 with four lenses 162, 164, 166, and 168. The lens 162 can be a biconcave lens, and the lenses 164 and 166 can be biconvex lenses. The lens 162 and 164 together can form a doublet. While FIGS. 4a and 4b show particular types of lenses in particular combinations, it is understood that any suitable lens types and combinations thereof can be implemented in the ocular lens 160 such that, together, they guide light beam 146 through the macular contact lens 150 and into the procedure eye 110, and generate the intermediate image plane 152. The individual components of the ocular lens 160 can be spaced from or in contact with one another. The individual components of the ocular lens 160 can be integrated into a single assembly or can be separate components. Component(s) of the ocular lens 160 can have a fixed focal length. Component(s) of the ocular lens 160 can also have a variable focal length. For example, one or more of the lenses 162, 164, and/or 166 (FIG. 4b) can be implemented as a zoom lens, a liquid crystal lens, and other suitable variable focal length lens. With the variable focal length lens(es), the field of view and lateral resolution for scanning with the light beam 146 and direct viewing with the microscope 120 can be adjusted based on the operator's preferences. In some embodiments, the reduction lens 170 can include one or more lenses with a variable focal length.

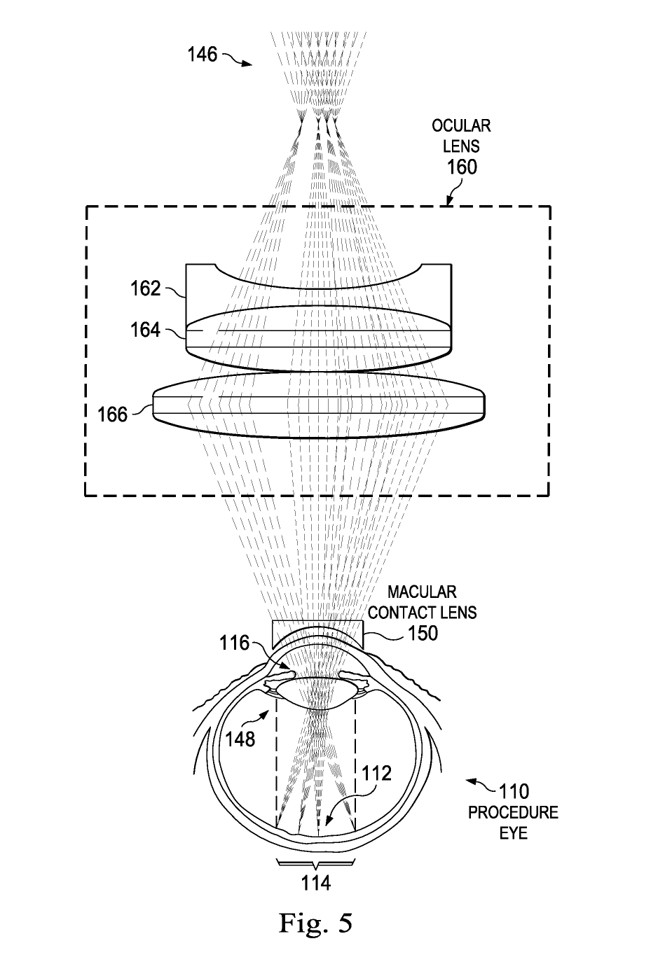

FIG. 5 illustrates a portion of the ophthalmic visualization system 100 that implements the ocular lens 160 of FIG. 4a. The light beam 146 can be guided by the lenses 162, 164, and 166 of the ocular lens 160 through the macular contact lens 150. In that regard, the lenses 162 and 164 cause the light beam 146 to diverge. The lens 166 causes the light beam 146 to converge. The light beam 146 can be focused at the target region 112 with the pivot point 148 at the pupil 116. Compared to when the ocular lens 160 and reduction lens 170 is not positioned in the optical path (FIG. 2), the light beam 146 can reach a wider area of the target region 112. Also, the total lens aberration, with the addition of the ocular lens 160 and/or the reduction lens 170, can be decreased compared to the lens aberration with the macular contact lens 150 only.

FIG. 6 illustrates a portion of the ophthalmic visualization system 100 associated with the light reflected and/or scattered from the procedure eye 110 and received at the surgical microscope 120. The intermediate image plane 152 formed by the light and the focus plane 122 of the surgical microscope 120 are coplanar. The intermediate image plane 152 and the focus plane 122 are located relatively closer to the surgical microscope 120 when the ocular lens 160 and the reduction lens 170 are positioned between the procedure eye 110 and the surgical microscope 120. In that regard, the ocular lens 160 can be configured to generate an image of the procedure eye 110 at the intermediate plane 152, which is positioned closer to the surgical microscope 120. When the ocular lens 160 and the reduction lens 170 are not positioned in the optical path, as shown in FIG. 2, the surgical microscope 120 is imaging directly on the procedure eye 110, and the focus plane 122 is positioned behind the macular contact lens 150 and along the target region 112, which is relatively farther from the surgical microscope 120. Referring again to FIG. 6, providing the ocular lens 160 in the optical path can move the intermediate image plane 152 between about 10 mm and about 200 mm, between about 20 mm and about 100 mm, and between about 50 mm and about 150 mm closer to the surgical microscope 120.

In order to preserve a clear image of the target region 112 through the surgical microscope 120, the reduction lens 170 can be configured to align the focus plane 122 with the intermediate image plane 152. For example, the reduction lens 170 can move the focus plane 122 by a distance corresponding to the distance that the intermediate image plane 152 is shifted by the ocular lens 160. For example, the reduction lens 170 can move the focus plane 122 between about 10 mm and about 200 mm, between about 20 mm and about 100 mm, and between about 50 mm and about 150 mm closer to the surgical microscope 120. Positioning the reduction lens 170 between the surgical microscope 120 and the ocular lens 160 can make the focus plane 122 and the intermediate image plane 152 coplanar without requiring an operator to adjust the focus controls of the surgical microscope 120 or changing the distance between procedure eye 110 and the surgical microscope 120. It is understood that one or more of any suitable lens types and combinations thereof can be implemented in the reduction lens such that, together, they align the focus plane 122 with the intermediate image plane 152.

While providing the ocular lens 160 and the reduction lens 170 in the optical path shifts the intermediate image plane 152 and the focus plane 122, the ocular lens 160 and the reduction 170 can be selected such that the magnification remains the same as when only the macular contact lens 150 is in place. That is, the size of the physiological features in the target region 112 can remain the same when the operator views the target region 112 with the ocular lens 160 and the reduction lens 170 compared to when only the macular contact lens 150 is in place. In this manner, the operator continues to work in familiar conditions with direct visualization while scanning of the light beam 146 across a wider field of the target region 112 is possible.

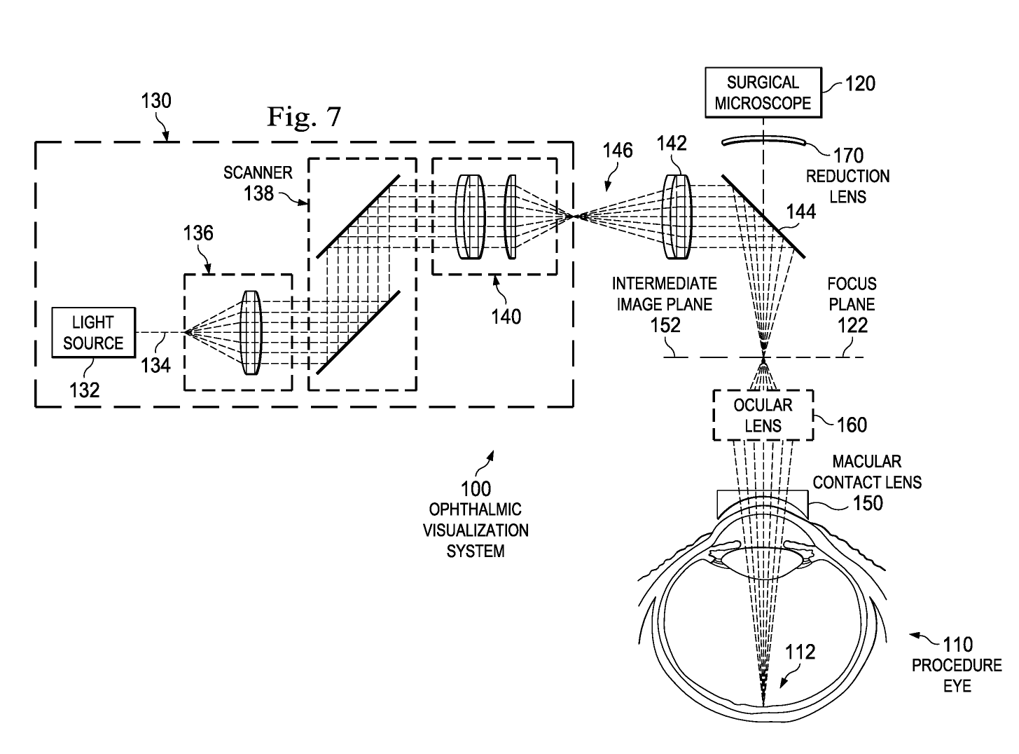

FIG. 7 illustrates an embodiment of the ophthalmic visualization system 100 in which the light beam 146 can be guided into the optical path between the surgical microscope 120 and the procedure eye 110 below the reduction lens 170. In that regard, the beam coupler 144 can be positioned between the reduction lens 170 and the ocular lens 160. An advantage of this arrangement can be the minimization of optical elements through which the light beam 146 travels, which can optimize performance for imaging/treatment/illumination beam delivery.

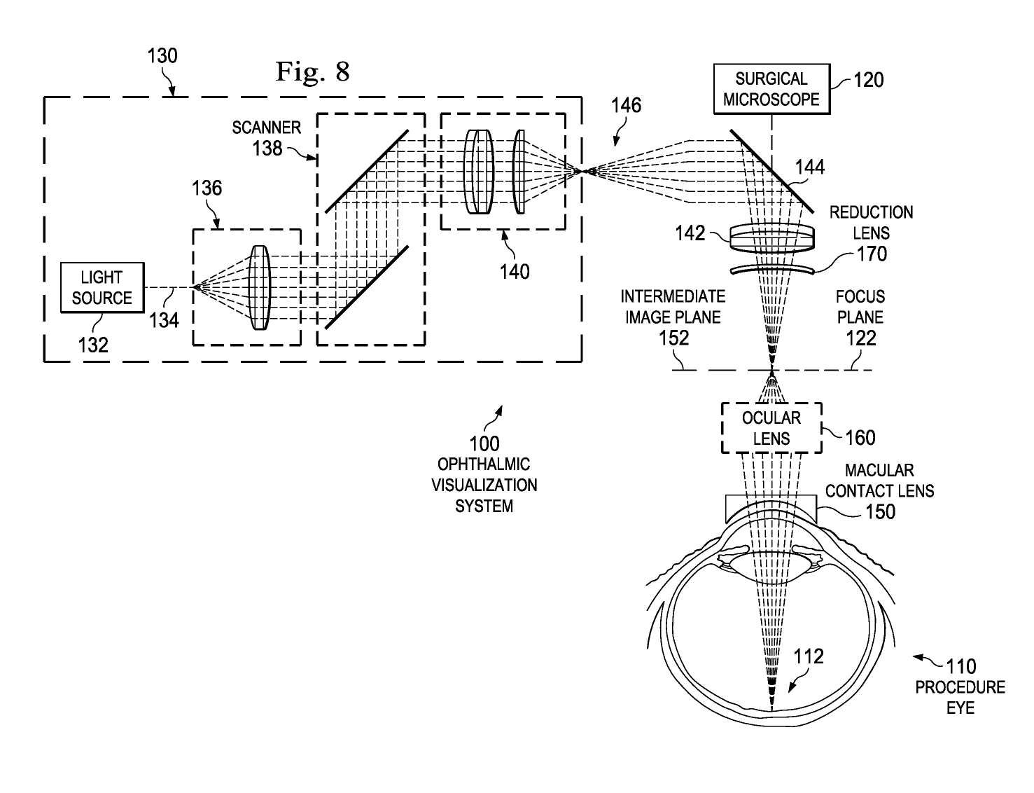

FIG. 8 illustrates an embodiment of the ophthalmic visualization system 100 in which the objective lens 142 can be shared by the surgical microscope 120 and the beam delivery system 130. The objective lens 142 can be shared, for example, when the beam delivery system 130 is integrated with the surgical microscope 120. In that regard, the objective lens 142 can be positioned between the beam coupler 144 and the procedure eye 110. For example, with the reduction lens 170 positioned between the objective lens 142 and the ocular lens 160 (as illustrated in FIG. 8), the objective lens 142 can be positioned between the beam coupler 144 and the reduction lens 170.

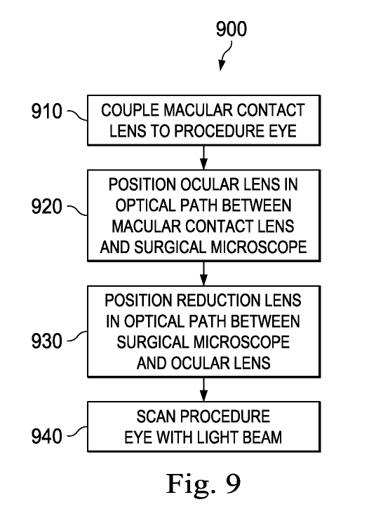

FIG. 9 illustrates a flow diagram of a method 900 of visualizing a procedure eye in an ophthalmic procedure. The steps of the method 900 can be better understood with reference to FIG. 1. As illustrated, the method 900 includes a number of enumerated steps, but embodiments of the method 900 may include additional steps before, after, and in between the enumerated steps. In some embodiments, one or more of the enumerated steps may be combined, omitted, or performed in a different order.

The method 900 can include, at step 910, coupling the macular contact lens 150 to the procedure eye 110. The procedure eye 110 can be positioned in the optical path of surgical microscope 120 and/or the beam delivery system 130. The method 900 can include, at step 920, positioning the ocular lens 160 in the optical path between the macular contact lens 150 and the surgical microscope 120. An intermediate image plane 152 associated with light reflected from the procedure eye 110 can be generated between the procedure eye 110 and the surgical microscope 120. The method 900 can include, at step 930, positioning the reduction lens 170 in the optical path between the surgical microscope 120 and the ocular lens 160. The focus plane 122 of the surgical microscope 120 can be aligned with the intermediate image plane 152. The step 930 can be performed before the step 920 and vice versa. The method 900 can include positioning the ocular lens 160 and the reduction lens 170 relative to one another such that the intermediate image plane 152 and the focus plane 122 are coplanar without either changing the distance between the surgical microscope 120 and the procedure eye 110, or refocusing the optics of the surgical microscope 120.

When the ocular lens 160 and the reduction lens 170 are integrated into an optical block, the method 900 can include selectively positioning the optical block within the optical path with the optical block movably coupled to the surgical microscope 120. For example, the steps 920 and 930 can be combined. When the ocular lens 160 and the reduction lens 170 are separate components, the method 900 can include selectively positioning the ocular lens 160 within the optical path with the ocular lens 160 movably coupled to at least one of the surgical microscope 120 and the reduction lens 170. The method 900 can also include selectively positioning the reduction lens 170 within the optical path with the reduction lens 170 movably coupled to at least one of the surgical microscope 120 and the ocular lens 160.

The method 900 can include, at step 940, scanning the procedure eye 110 with the light beam 146. Scanning the procedure eye 110 can include guiding the light beam 146 through the macular contact lens 150 and into the procedure eye 110 using the ocular lens 160. The method 900 can include generating the light beam 146, such as a diagnostic light beam or a treatment light beam using the light source 132. The method 900 can include guiding the light beam 146 from the light source 132 to the scanner 138. The method 900 can include scanning the light beam 146 using the scanner 138. The method 900 can include redirecting the scanned light beam 146 using the beam coupler144. Redirecting the scanned light beam 146 can include redirecting the scanned light beam 146 into the optical path between the surgical microscope 120 and the procedure eye 110 to scan the procedure eye 110. The method 900 can include selectively removing the ocular lens 160 and the reduction lens 170 from the optical path. That is, the operator can use the ophthalmic visualization system 100 for direct visualization only through microscope and/or for combined scanning and direct visualization.

Embodiments as described herein can provide devices, systems, and methods that facilitate a simplified workflow for ophthalmic procedures including a macular contact lens that provides both direct viewing of a target region with a surgical microscope as well as scanning with a diagnostic or a treatment light beam. The examples provided above are exemplary only and are not intended to be limiting. One skilled in the art may readily devise other systems consistent with the disclosed embodiments which are intended to be within the scope of this disclosure. As such, the application is limited only by the following claims.

1. An ophthalmic visualization system, comprising:
an ocular lens configured to be positioned in an optical path between a macular contact lens coupled to a procedure eye and a surgical microscope, wherein the ocular lens is configured to
guide a light beam through the macular contact lens and into the procedure eye; and
generate an intermediate image plane associated with light reflected from the procedure eye, the intermediate image plane being positioned between the procedure eye and the surgical microscope; and
a reduction lens positioned in the optical path between the surgical microscope and the ocular lens, wherein the reduction lens is configured to align a focus plane of the surgical microscope with the intermediate image plane.

2. The system of claim 1, wherein:
the ocular lens is configured to guide the light beam through the macular contact lens and into the procedure eye such that a pivot point of the light beam is located at or near a pupil of the procedure eye.

3. The system of claim 1, wherein:
the reduction lens is positioned relative to the ocular lens in the optical path such that the intermediate image plane and the focus plane are coplanar without either changing the distance between the surgical microscope and the procedure eye or refocusing optics of the surgical microscope.

4. The system of claim 1, wherein:
the ocular lens and the reduction lens are separate components such that each one of ocular lens and the reduction lens is movably coupled to at least one of the surgical microscope and the other of the ocular lens and the reduction lens.

5. The system of claim 1, wherein:
the ocular lens and the reduction lens are integrated into an optical block; and
the optical block is movably coupled to the surgical microscope such that the optical block is selectively positionable within the optical path.

6. The system of claim 1, wherein:
at least one of the ocular lens and the reduction lens has a variable focal length.

7. The system of claim 1, further comprising:
the macular contact lens coupled to the procedure eye.

8. The system of claim 1, wherein the light beam is at least one of:
a treatment light beam, a diagnostic light beam, and an illumination light beam.

9. The system of claim 8, wherein:
the light beam is part of a diagnostic imaging system.

10. The system of claim 9, wherein the diagnostic imaging system is at least one of:
an optical coherence tomography (OCT) system, a multispectral imaging system, a fluorescence imaging system, a photo-acoustic imaging system, a confocal scanning imaging system, and a line-scanning imaging system.

11. The system of claim 8, wherein:
the light beam is part of a treatment beam delivery system.

12. The system of claim 11, wherein the treatment beam delivery system is at least one of:
a photocoagulation system, a photodynamic therapy system, and a retinal laser treatment system.

13. The system of claim 8, wherein:
the light beam is part of an illumination beam delivery system.

14. The system of claim 13, wherein the illumination beam delivery system is configured to output at least one of:
a red illumination light, a blue illumination light, a green illumination light, a visible illumination light, a near infrared illumination light, and an infrared illumination light.

15. A method visualizing a procedure eye in an ophthalmic procedure, the method comprising:
positioning an ocular lens in an optical path between a macular contact lens coupled to the procedure eye and a surgical microscope such that an intermediate image plane associated with light reflected from the procedure eye is generated between the procedure eye and the surgical microscope;
positioning a reduction lens in the optical path between the surgical microscope and the ocular lens such that a focus plane of the surgical microscope is aligned with the intermediate image plane; and
scanning the procedure eye with a light beam including guiding the light beam through the macular contact lens and into the procedure eye using the ocular lens.

16. The method of claim 15, wherein at least one of positioning the ocular lens and positioning the reduction lens includes:
positioning the ocular lens and the reduction lens relative to one another such that the intermediate image plane and the focus plane are coplanar without either changing the distance between the surgical microscope and the procedure eye or refocusing optics of the surgical microscope.

17. The method of claim 15, wherein:
the ocular lens and the reduction lens are integrated into an optical block; and
positioning the ocular lens and positioning the reduction lens includes selectively positioning the optical block within the optical path with the optical block movably coupled to the surgical microscope.

18. The method of claim 15, wherein:
the ocular lens and the reduction lens are separate components; and
positioning the ocular lens includes selectively positioning the ocular lens within the optical path with the ocular lens movably coupled to at least one of the surgical microscope and the reduction lens; and
positioning the reduction lens includes selectively positioning the reduction lens within the optical path with the reduction lens movably coupled to at least one of the surgical microscope and the ocular lens.

19. The method of claim 18, further comprising:
selectively removing the ocular lens and the reduction lens from the optical path.

20. The method of claim 15, further comprising:
coupling the macular contact lens to the procedure eye.

21. The method of claim 15, further comprising:
generating the light beam using a light source;
guiding the light beam from the light source to a scanner;
scanning the light beam using the scanner;
redirecting the scanned light beam using a beam coupler, including redirecting the scanned light beam into the optical path between the surgical microscope and the procedure eye to scan the procedure eye.

22. The method of claim 21, wherein generating a light beam includes:
generating at least one of a diagnostic light beam, a treatment light beam, and an illumination light beam.

23. The method of claim 22, wherein:
the light source and the beam scanner are part of at least one of a diagnostic imaging system, a treatment beam delivery system, and an illumination beam delivery system.

24. The method of claim 23, wherein the diagnostic imaging system is at least one of:
an optical coherence tomography (OCT) system, a multispectral imaging system, a fluorescence imaging system, a photo-acoustic imaging system, a confocal scanning imaging system and a line-scanning imaging system.

25. The method of claim 23, wherein the treatment beam delivery system is at least one of:
a photocoagulation system, a photodynamic therapy system, and a retinal laser treatment system.

26. The method of claim 23, wherein the illumination beam delivery system is configured to output at least one of:
a red illumination light, a blue illumination light, a green illumination light, a visible illumination light, a near infrared illumination light, and an infrared illumination light.
